(19) United States
(12) Patent Application Publication
(10) Pub. No.: US 2018/0129783 A1
(43) Pub. Date: May 10, 2018

(54) Distant Asthma Assessment And Treatment System

(71) Applicant: dox4all, Inc., Loveland, CO (US)

(72) Inventor: Timothy Ryschon, Loveland, CO (US)

(73) Assignee: dox4all,Inc., Loveland, CO (US)

(21) Appl. No.: 15/799,318

(22) Filed: Oct. 31, 2017

Related U.S. Application Data
(60) Provisional application No. 62/417,977, filed on Nov. 4, 2016.

(51) Int. Cl. G06F 19/00; A61B 5/091

(52) U.S. Cl. CPC G06F 19/34; A61B 5/087; G06F 19/322; A61B 5/091

(57) ABSTRACT

A computer implemented distant asthma assessment and treatment system including a processor communicatively coupled to a memory element including a computer program having an asthma condition assessment module executable to receive indications of an asthma condition of a patient from an asthma condition assessment device and further including a medical record update module executable to enter indications of the asthma condition in a medical record of the patient and an asthma treatment module executable to provide asthma treatment prescriptions to the patient based on indications of the asthma conditions entered into the medical record and an asthma condition tracking module which receives indications of asthma condition over a duration of time processable by the asthma treatment control module to alter subsequent asthma treatment prescriptions for treatment of indications of the asthma condition.

### I. FIELD OF THE INVENTION

A computer implemented distant asthma assessment and prescribing system including a processor communicatively coupled to a memory element including a computer program having an asthma condition assessment module executable to receive indications of an asthma condition of a patient from an asthma condition assessment device and further including a medical record update module executable to enter indications of the asthma condition in a medical record of the patient and a clinician module executable to provide asthma treatment prescriptions to the patient based on indications of the asthma conditions entered into the medical record and an asthma condition tracking module which receives indications of asthma condition over a duration of time processable by the asthma treatment control module to alter subsequent asthma treatment prescriptions for treatment of indications of the asthma condition.

### II. SUMMARY OF THE INVENTION

A broad object of the invention can be to provide a computer implemented distant asthma assessment and treatment system (also referred to as the “system”) in the form of a server and a server processor communicatively coupled to a server memory containing a computer program having computer-executable instructions which can be served in whole or in part to depict on the display surface of a computing device of a patient a graphical user interface including a setup menu in which a patient can subscribe and log on to the system, enter a patient profile, and release patient medical records to a clinician, and a run menu which receives indications of an asthma condition of a patient whether manually or wirelessly from an asthma condition assessment device, depicts asthma treatment prescriptions or information to the patient based on the indications of the asthma conditions, receives indications of appointments for and payment of clinician services in providing prescriptions or information to the patient and depicts on the display surface of computing device of a clinician a graphical user interface including one or more of: the patient profile, released patient medical records, indications of the asthma condition of patient, indications of appointments and payments for clinician services, and fields to enter asthma treatment prescriptions and information based on indications of asthma conditions.

A broad object of the invention can be to provide a computer implemented distant asthma assessment and treatment system in the form of a server and a server processor communicatively coupled to a server memory containing a computer program including one or more of: a sign up and log in module executable to allow a patient to subscribe and log in to the system, a medical information module which allows a patient to release and update medical records and a clinician to access the medical records of a patient, an asthma condition assessment module executable to receive indications of an asthma condition of a patient from an asthma condition assessment device, an asthma treatment module executable to provide asthma treatment prescriptions or information based on indications of the asthma conditions entered into the medical record, an asthma condition tracking module which receives indications of an asthma condition over a duration of time processable by the asthma treatment control module to alter subsequent asthma treatment prescriptions for treatment of indications of the asthma condition, and an appointment and payment module which allows a patient to make an appointment with a clinician and make payment for clinician services in treatment of indications of an asthma condition.

Naturally, further objects of the invention are disclosed throughout other areas of the specification, drawings, photographs, and claims.

### III. BRIEF DESCRIPTION OF THE DRAWINGS

FIG. 1 is an illustration of method of using a particular embodiment of the computer implemented distant asthma assessment and prescribing system.

FIG. 2 is a block diagram of a particular embodiment of the computer implemented distant asthma assessment and prescribing system.

FIG. 3 is an illustration of a particular embodiment of a user interface depicting a sign up-login menu.

FIG. 4 is an illustration of a particular embodiment of a user interface depicting a setup menu which allows input of user information.

FIG. 5 is an illustration of a particular embodiment of a user interface depicting a medical records release menu.

FIG. 6 is an illustration of a particular embodiment of a user interface depicting the medical records or medical history of a user.

FIG. 7 is an illustration of a particular embodiment of a user interface depicting an asthma condition assessment menu.

FIG. 8 is an illustration of a particular embodiment of a user interface depicting an asthma condition assessment questionnaire.

FIG. 9 is an illustration of a particular embodiment of a user interface depicting a peak flow test procedure.

FIG. 10 is an illustration of a particular embodiment of a user interface depicting an asthma condition tracking menu.

FIG. 11 is an illustration of a particular embodiment of a user interface depicting a prescription menu including one or more prescriptions.

FIG. 12 is an illustration of a particular embodiment of a user interface depicting an appointment menu.

FIG. 13 is an illustration of a particular embodiment of a user interface depicting a payment menu.

FIG. 14 is an illustration of a particular embodiment of a user interface depicting a clinician menu.

FIG. 15 is an illustration of a particular embodiment of a user interface depicting a geo-spatial location menu.

FIG. 16 is an illustration of a particular embodiment of a user interface depicting a prescription ordering menu.

### IV. DETAILED DESCRIPTION OF THE INVENTION

### General Overview of System.

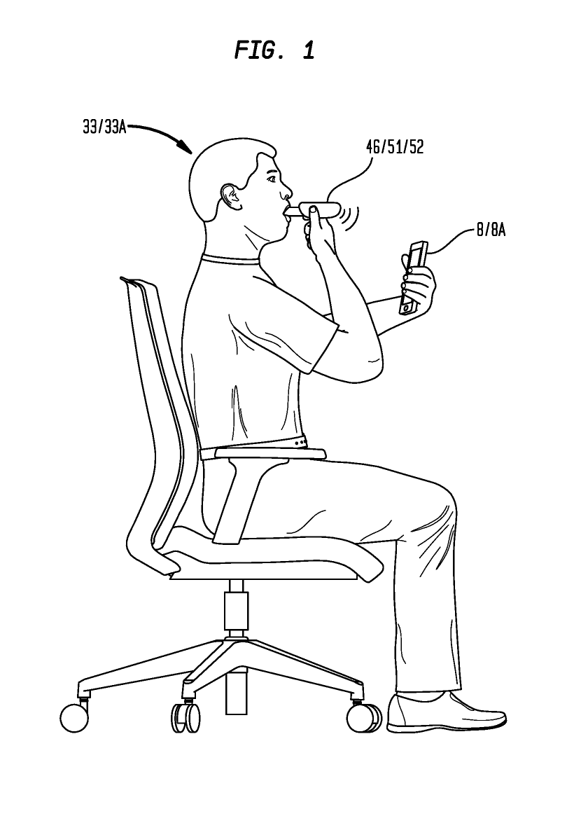

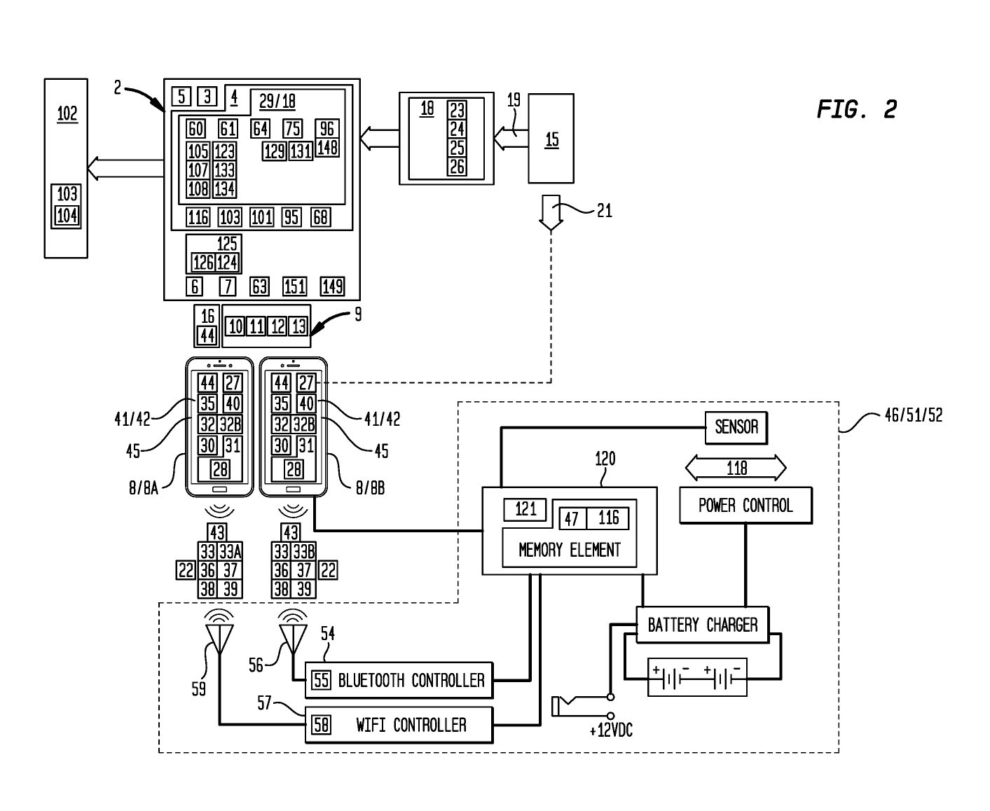

Now referring primarily to FIGS. 1 and 2, which illustrate a computer implemented asthma assessment and prescription system (1)(also referred to as the “system”) which may be distributed on one or more servers (2), each having a server processor (3), a server memory (4), a server operating system (5), a server input/output interface (6), and a server network interface (7) operatively communicating with one or more computing devices (8) via a network (9) including one or more of a public network (10), such as the Internet (11), a cellular-based wireless network (12), or a local network (13)(also individually or collectively referred to as a “network (9)”). The system (1) can, but need not necessarily, further include a location identification server (14) operably coupled to a global positioning system (15) (“GPS”).

The network (9) supports a plurality of communication resources (16)(along with other communication resources made available in the future) to afford recording, transmission, or reproduction of images (whether still or moving images), sound relating to acoustical, mechanical or electrical frequencies, electronic mail, instant messaging, text messaging (such as short message service) multimedia messaging (such as multimedia message service) attributable to the execution of self-contained programs or pieces of software designed to fulfill particular purposes (also referred to as “applications” (17)), such as web applications, online applications, mobile applications, or the like, downloadable to one or more computing devices (8).

The location identification server (14) contains a global positioning application (18)(“GPS application”) capable upon execution of receiving and decoding a global positioning signal (19) generated by the GPS (15). The term “global positioning system (15)”, for the purposes of this invention, means a plurality of earth-orbiting satellites (20) each transmitting a satellite positioning signal (21) continuously towards the Earth (22), that enables a coordinate location (23), including a longitude (24), a latitude (25) or altitude (26) of a global positioning signal receiver (27) on or near the surface of the Earth (22) to be accurately estimated.

### The Asthma Assessment and Prescription Program.

Again, referring primarily to FIGS. 1 and 2, the server (2) can include a server processor (3) communicatively coupled to the server memory (4) containing an asthma assessment and prescribing program (28) (also referred to as the “program (28)”) which is described below in the general context of computer-executable instructions such as program modules (29) which utilize routines, programs, objects, components, data structures, or the like, to perform particular functions or tasks or implement particular abstract data types, however, it is not intended that any embodiment be limited to a particular set of computer-executable instructions or protocols.

As to particular embodiments, the program (28) can be downloaded in whole from the server (2) to a first computing device (8A) having a computing device processor (30) communicatively coupled to a computing device memory element (31) to discretely confer all of the program functions (32) of the program (28) to the first computing device (8A) for use only by a first user (33)(which can be a patient (33A)), or as to other embodiments, the program (28) can be downloaded in part to a first computing device (8A) for use by the first user (33) and in part to a second computing device (8B) for use by a second user (34)(which can be clinician (34A)) to discretely confer a first portion of the program functions (32A) of the program (28) to the first computing device (8A) and discretely confer a second portion of the program functions (32B) of the program (28) to the second computing device (8B) for integrated or coordinated use of the first computing device (8A) by the first user (33) and the second computing device (8B) by the second user (34). The program (28) can operatively communicate with the server (2) over the network (9) to communicatively couple the first computing device (8A) with the server (2) to coordinate or pair operation of the first computing device (8A) with operation of a second computing device (8B).

As to particular embodiments, the first computing device (8A) or the second computing device (8B) can, but need not necessarily, download the GPS application (18), which may be operatively coupled to the location identification server (14). As to embodiments of the first or the second computing device (8A)(8B) including GPS functions (35), the program (28) in coordination with the GPS application (18) can be executed to retrieve or receive a coordinate location (23) of the first or second computing device (8A)(8B) from the location identification server (14). The program (28) in coordination with the GPS application (18) can further receive a coordinate location (23) of the first computing device (8A) or the second computing device (8B), which may include a current location (36) at a current time (37) or expected location (38) at a future time (39).

The term “computing device (8)” for the purposes of this invention means desktop or mobile computer devices which include a computing device processor (30) communicatively coupled to a computing device memory element (31) which can contain in whole or in part the program (28) or can perform browser based processing in downloaded computing device content (44) and without sacrificing the breadth of the foregoing includes personal computers, slate, tablet or pad computers, and cellular telephones or camera/cell phones, and programmable consumer electronics.

While embodiments of the program (28) are described in the general context of computer-executable instructions such as program modules (29) which utilize routines, programs, objects, components, data structures, or the like, to perform particular functions or tasks or implement particular abstract data types, it is not intended that any embodiments be limited to a particular set of computer-executable instructions or protocols.

As to particular embodiments, the program (28) can also be loaded to and contained in whole or in part in the computing device memory element (31) of the first or second computing devices (8A)(8B)(or a plurality of computing devices (8)) from one or more of: a computer disk, universal serial bus flash drive, or other computer readable medium, without communication with a server (2) or use of any network (9). The term “machine readable medium” for the purposes of this invention means any medium capable of non-volatile storage of machine readable code in a format readable by a mechanical device and without sacrificing the breadth of the forgoing a magnetic media, an optical media, a non-volatile memory, or the like, or combinations thereof.

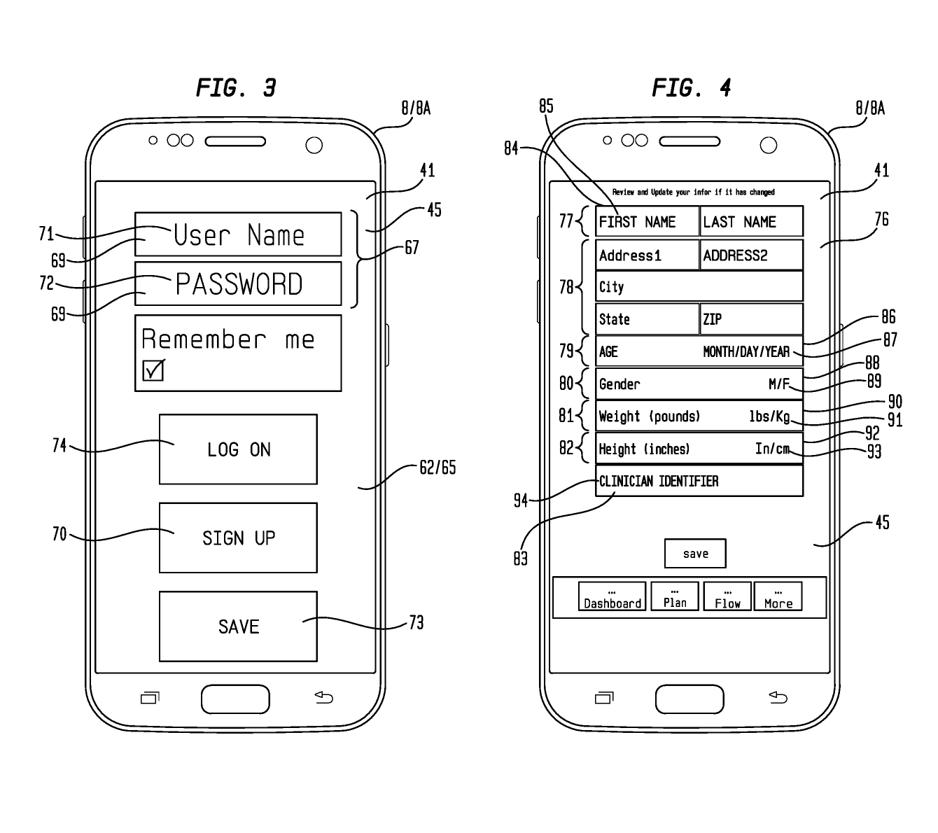

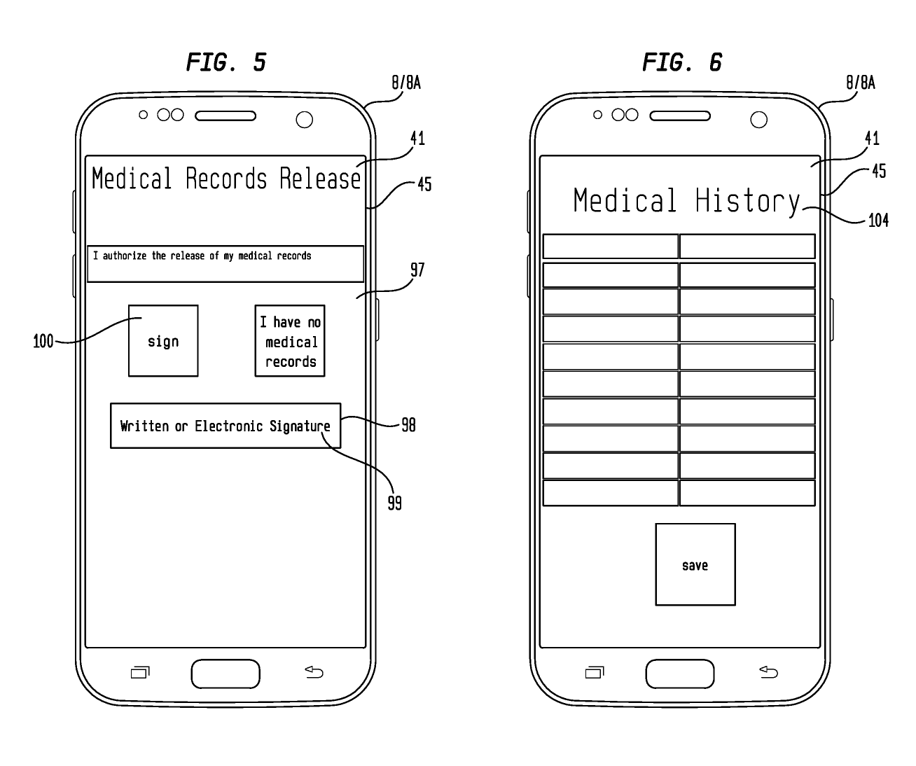

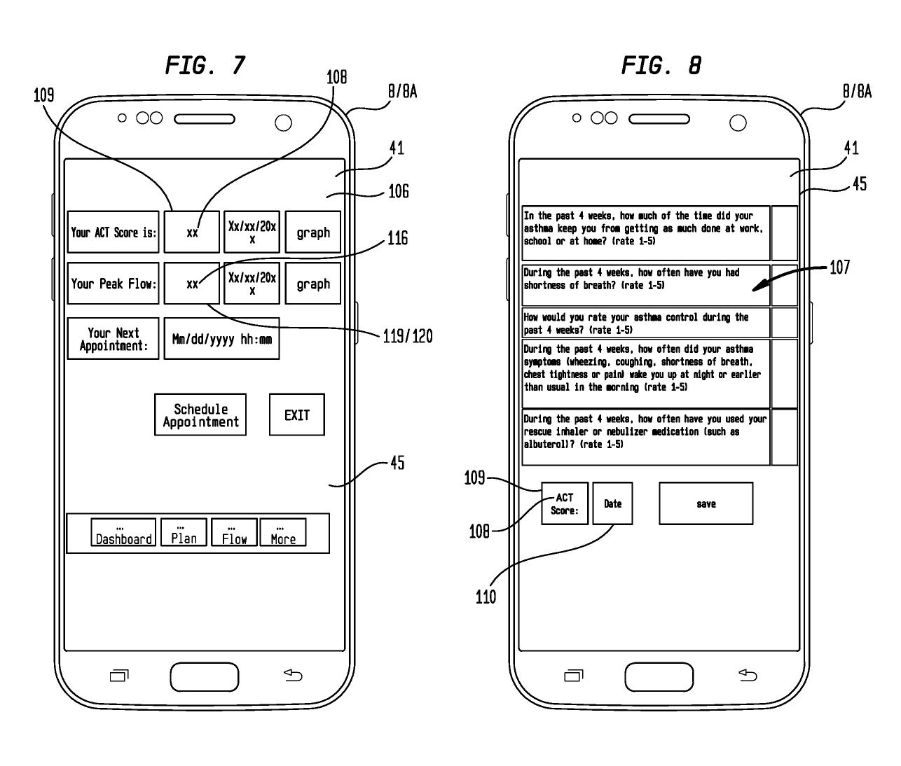

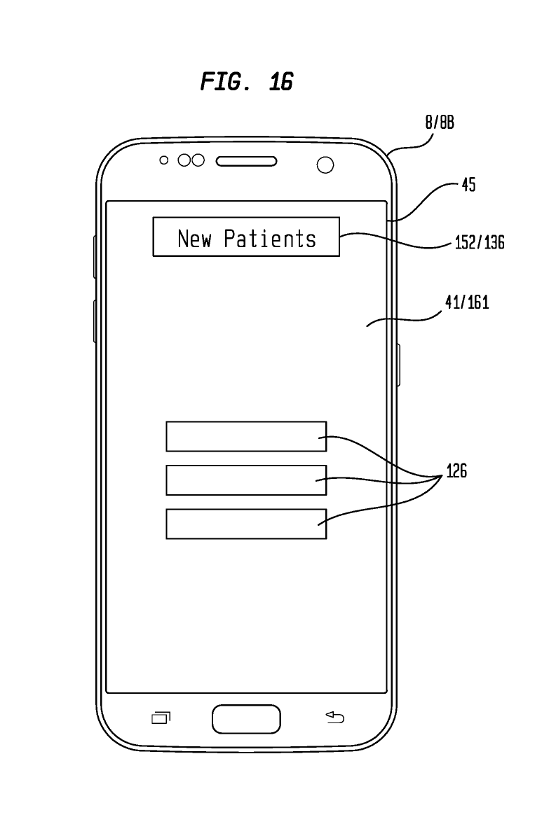

Again, referring primarily to FIG. 1, each of the one or more computing devices (8) can, but need not necessarily, include an Internet browser (40) (also referred to as a “browser”) such as Microsoft's INTERNET EXPLORER®, GOOGLE CHROME®, MOZILLA®, FIREFOX®, or the like, which functions to download and render computing device content (44) formatted in “hypertext markup language” (HTML). In this environment, the one or more servers (2) can contain the program (28) which implements the most significant portions of one or more graphical user interface(s)(41) depicted on the display surface (45) of a computing device (8) including one or more menus (42) including a combination of text and symbols to represent options selectable by user command (43) to execute one or more program functions (32) of the program (28). As to these embodiments, the one or more computing devices (8) can use the browser (40) to depict downloaded computing device content (44) and to relay selected user commands (43) back to the one or more servers (2). The one or more servers (2) can respond by formatting new menus (42) for the respective graphical user interfaces (41) (as shown in the illustrative examples of FIGS. 3 through 16 further described below).

Again, referring primarily to FIGS. 1 and 2, in other embodiments, the one or more servers (2) can be used primarily as sources of computing device content (44), with primary responsibility for implementing the graphical user interface (41) being placed upon each of the one or more computing devices (8). As to these embodiments, each of the one or more computing devices (8) can run the appropriate portions of the program (28) implementing the corresponding program functions (32) including but not limited to the depiction of the graphical user interfaces (41).

### The Asthma Assessment Device.

Now referring primarily to FIGS. 1 and 2, embodiments of the system (1) can further include an asthma assessment device (46). The term “asthma condition assessment device (46)” for the purposes of this invention means any device capable of generating or which generates asthma condition metrics (47) correlatable with an asthma condition (48) which by user command (43) can be entered into a graphical user interface (41) depicted on the display surface (45) of the first or second computing device (8A)(8B) as indications of an asthma condition (49) and without sacrificing the breadth of the foregoing a device, whether or not configured for wired or wireless pairing with the first or the second computing device (8A)(8B), useful in performing a lung function test (50), including as illustrative examples:

A spirometer (51) which measures the air capacity of the lungs. Spirometry measures one or more of forced vital capacity (“FVC”), forced expiratory volume (FEV) at timed intervals of 0.5, 1.0 (FEV1), 2.0, (FEV2) and 3.0 (FEV3) seconds, forced expiratory flow 25-75% (“FEF 25-75”) and maximal voluntary ventilation (“MVV”). Forced vital capacity (FVC) is the volume of air that can forcibly be blown out after full inspiration, measured in liters. The forward vital capacity (FVC) test varies slightly depending on the equipment used. FEV1 is the volume of air that can forcibly be blown out in one second, after full inspiration. Maximum voluntary ventilation (MVV) is a measure of the maximum amount of air that can be inhaled and exhaled within one minute.

A peak flow meter (52) which measures the velocity of a forced expiratory volume or how quickly the patient can blow air out of the lungs. However, as to particular embodiments, the peak flow meter (52) can be configured to measure the forced inspiratory volume or a combination of the forced expiratory volume and forced inspiratory volume. As to particular embodiments of the peak flow meter (52) respiratory volumes including both normal or forced expiratory volume and inspiratory volumes can be measured and converted to asthma condition metrics useful in assessing the asthma condition (48) and prescribing medicaments to alleviate the asthma condition (48).

The term “asthma condition (48)” for the purposes of this invention means any physical or mental feature or function relating to asthma of a patient (33A).

The term “asthma condition metrics” for the purposes of this invention means the quantitative value (47A), which can but need not be a numeric value, obtained by an asthma condition assessment device (46) in measuring an asthma condition (48) and without sacrificing the breadth of the foregoing a numeric value obtained in performance of a lung function test (50).

The term “patient (33A)” for the purposes of this invention means an animal (whether human or non-human) having an asthma condition (48).

The term “clinician (34A)” for the purposes of this invention means a physician of medicine or person having direct contact with and responsibility for the patient (33A).

The term “wireless” for the purposes of this invention means using electromagnetic waves (53) rather than some form of wire to carry a signal over all or a part of a communication path and without sacrificing the breadth of the foregoing can include BLUETOOTH® enabled in the ISM band from 2400-2480 MHz or Wi-Fi® enabled in one or more channels in the 2.4 GHz band, or the like, or combinations thereof, for the exchange of data over the communication path.

As to particular embodiments, an asthma condition assessment device (46) can, but need not necessarily, include a BLUETOOTH® controller (54) (as shown in the illustrative example of FIG. 2 a Texas Instruments CC2540 BLUETOOTH® System-on-Chip) including the associated BLUETOOTH® transceiver (55) and BLUETOOTH® antenna (56). As to particular embodiments, the asthma condition assessment device (46) can, but need not necessarily, include a WiFi® controller (57) and the associated Wi-Fi® receiver (58) and Wi-Fi® antenna (59). As to other embodiments, the asthma condition assessment device (46) can provide both a BLUETOOTH® controller (54) and a Wi-Fi® controller (57) including the associated transceivers (55)(58) and antennas (56)(59).

Wireless embodiments of asthma condition assessment devices (46) can be placed in the active condition by a first user (8A) such that the asthma condition assessment device (46) seeks a connection with a first computing device (8A) containing or having access to the program (28) within the range of the transceiver (54)(58)(or vice versa). The program (8) can cause pairing of the first computing device (8A) with the asthma condition assessment device (46). The asthma condition assessment device (46) can be used in accordance with the manufacturer's instructions and transmits one or more asthma condition metrics (47) to first or second computing devices (8A)(8B).

As to embodiments of asthma condition assessment devices that are not BLUETOOTH® or Wi-Fi® enabled, the patient (33A) can manually record or enter into a menu (42) of a graphical user interface (41) the asthma condition metric (47) obtained by the asthma condition assessment device (46) in performance of a lung function test (50)

### The Graphical User Interface.

Now referring primarily to FIGS. 2 through 16, the program (28) in part includes a user interface module (60) executable to generate a user interface which can, but need not necessarily, be a graphical user interface (41) displayed on the display surface (45) of a computing device (8) which allows a first user (33) (for example a patient (33A)) or second user (34) (for example a clinician (34A)) to execute by user command (43) one or more program functions (32) of the program (28). The user command (43) can, as illustrative examples, be: selection of one or more control icon(s), entry of text into one or more fillable fields, voice command, keyboard stroke, mouse button point and click, touch on a touch screen, or otherwise, or combinations thereof (individually and collectively referred to as a “user command”).

### The Signup Module.

Now referring primarily to FIGS. 2 and 3, embodiments of the program (28) can, but need not necessarily, include a sign up module (61) which upon execution depicts a sign up menu (62) on the display surface (45) of a computing device (8) which by user command (43) allows the first user (33) to create an account (63) which allows the first user (33) to authenticate to the system (1) and potentially receive authorization to access resources (16) provided by or connected to the system (1) and to load the program (28) in whole or in part to the first computing device (8A).

### The Login Module.

Now referring primarily to FIGS. 2 and 3, embodiments of the program (28) can, but need not necessarily, include a login module (64) which upon execution depicts a login menu (65) which by user command (43) allows the first user (33) to log in to an account (63). To log in to an account (63), a user (33) is typically required to authenticate oneself with a user identifier (67) or other credentials for the purposes of accounting, security, logging, and resource management. Once the user (33) has logged on, the system (1) will often use a user identifier (67) such as an integer to refer to them, rather than their user name, through a process known as identity correlation. As one illustrative example, in Unix® systems, the user name is correlated with the user identifier (67).

For the purposes of this invention the term “user identifier (67)” means integers or characters or combination thereof unique to a user (33)(33A)(34)(34A) which by operation of the login module (64) can be matched by the server (2) or computing device (8) to the stored data structures (68) of that user (33). In the illustrative example of FIG. 3, the log in module (64) depicts a user identifier (67) entered into a user identifier field (69).

As to particular embodiments, the sign up menu (62) and the log in menu (65) can be combined in one sign up login menu (62)(65)(as shown in the example of FIG. 3). As an example, a first user (33) can by interaction in the sign up/log in menu (62)(65) with a sign up button (70) cause the sign up module (61) to depict a plurality of user identifier fields (69) which are shown in the example of FIG. 3 “User Name” and “Password.” The first user (33) can enter a user name (71) and a user password (72) and by user command (34) (shown in the example of FIG. 3 as a “Save” button (73)) submit the user name (71) and password (72) to sign up in the system (1). Subsequently, the first user (33) can by user command (43) select the “Log On” button (74) and enter the user name (71) and password (69) to access the system (1).

### The Setup Module.

Now referring primarily to FIGS. 2 and 4, embodiments of the program (28) can, but need not necessarily, include a setup module (75) which by user command (43) allows settings or values to be assigned to the program (28) or the computing device (8) which are associated with the account (63) of the first user (33). Certain embodiments can, but need not necessarily, be provided with presets which provide settings or values automatically assigned by or to the program (28) outside of first user (33) intervention.

Now referring primarily to FIGS. 2 and 4, the setup module (75) can function to display a setup menu (76) on the display surface (45) of the first computing device (8A) of the first user (33) which by user command (43) allows input of one or more of first user's: name (77), address (78), age status (79), gender status (80), weight status (81), and height status (82) and prior treating physician (83). While the embodiment illustrated in FIG. 4 depicts a name identification field (84) in which a name identifier (85) can entered, an age status field (86) in which age status values (87) (month, day, year) can be selected or entered, a gender status field (88) in which gender status values (89)(male, female) can be entered or selected, a weight status field (90) in which weight status values (91)(pounds or kilograms) can be entered or selected, a height status field (92) in which height status values (93) can be entered or selected, and prior treating physician identification field (94) in which the name of the prior treating physician (83) can be selected or entered; this illustrative example is not intended to preclude depiction of additional or alternate status fields or status values relating to anatomical or physiological attributes of a first user (34) or medicaments taken by the first user (34) which may be relevant to the asthma condition (48) of the first user (33).

Now referring primarily to FIGS. 2 and 4, as to particular embodiments, the set up module (75) can further function to match the setup values input by user command (43) into the set up menu (76) with the user identifier (67) or account (63) to create a user profile (95) which can be stored remotely in the server memory (2) or locally in the computing device memory element (31).

### The Medical Records Module.

Now referring primarily to FIGS. 2 and 5 through 7, embodiments of the program (28) can, but need not necessarily, further include a medical records module (96) which can function to depict on the display surface (45) of the computing device (8) a medical records release menu (97) including a signature field (98) in which the first user (33) can provide an electronic signature or written signature (99). By entry of the electronic or written signature (99) in the signature field (98), and as to certain embodiments, further interaction with a confirmatory medical records release icon (100)(shown in the example as “Sign” button) the medical records module (96) functions to generate and save a medical records release (101) in the system (1). As to particular embodiments, the medical records module (96) can further function to access the pertinent medical records databases (102) to obtain and save the medical records (103) of the first user (33)(patient (33A)) in the system (1)(as shown in the example of FIG. 6, the medical records (103) can in part include the medical history (104) of the first user (33)).

### The Asthma Condition Assessment Module.

Now referring primarily to FIGS. 2 and 7 through 10, embodiments of the program (28) can, but need not necessarily, further include an asthma condition assessment module (105) executable to depict an asthma condition assessment menu (106) on the display surface (45) of the first computing device (8A). The asthma condition assessment menu (106) receives the indications of an asthma condition (49) of a first user (33)(patient (33A)).

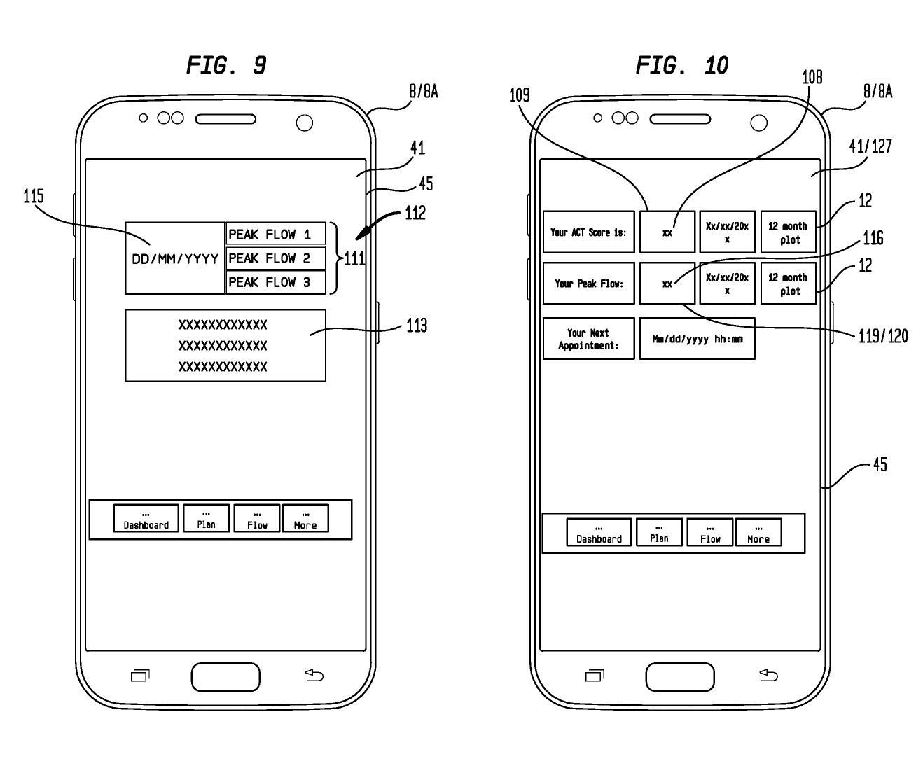

Now referring to primarily to FIGS. 8 and 9, as to particular embodiments, the asthma condition assessment module (105) can, but need not necessarily, depict an asthma condition assessment questionnaire (107), such as an Asthma Control Test® (“ACT”)(Quality Metric Incorporated) or an Asthma Control Questionnaire (“ACQ”) in the form a short questionnaire in which responses can be entered into the ACT or ACQ by user command (43) (as shown in the example of FIG. 9 by selection of clickable bullets). The ACT or ACQ numerically scores the responses and provides an asthma condition assessment questionnaire score (108) in an asthma condition questionnaire score field (109)(for example an ACT or ACQ score) along with an asthma condition assessment questionnaire test date (110) (ACT or ACQ test date) which can be entered by user command (43) or can be automatically entered by operation of the asthma condition assessment module (105) into an asthma condition assessment questionnaire score field (109) (ACT or ACQ score field) in the asthma condition assessment menu (107).

Now referring primarily to FIGS. 9 and 10, as to particular embodiments, the asthma condition assessment module (105) can function to receive indications of asthma condition (49) in the asthma condition assessment menu (106) from an asthma condition assessment device (46) subsequent to performance of a lung function test (50), as above described. The asthma condition assessment device (46) can be a flow meter (52) or a spirometer (51) and the lung function test (50) can be one or more peak flow tests (111). Now referring primarily to FIG. 10, in the illustrative example, the asthma condition assessment module (105) functions to depict a peak flow test menu (112) on display surface (45) of the first computing device (8A). The peak flow test menu (112) can include a peak flow test procedure (113) which the first user (33)(patient 33A) follows to perform one or more peak flow tests (111). As to particular embodiments, the first user (33)(patient 33A) can enter or select the manufacturer's product number (114) associated with the flow meter (52) or spirometer (51) and the asthma condition assessment module (105) can correspondingly function to depict manufacturer's peak flow test procedure (113) to perform the peak flow test (111) with that particular flow meter (52) or spirometer (51). The peak flow test menu (112) can further depict a calendar date (115) on which each of the one or more peak flow tests (111) are to be performed. Subsequent to performing each of the peak flow tests (111) in accordance with the peak flow test procedure (113), the flow meter (52) or spirometer (51) can provide a peak flow metric (116) corresponding to the peak flow (118) measured by the flow meter (52) or spirometer (51). As to embodiments of the peak flow meter (52) or spirometer (51) which can be communicatively paired with the first computing device (8A)(whether wired or wirelessly as above described), the peak flow metric (116) and the calendar date (115) on which the peak flow metric (116) was generated can be automatically entered into the peak flow test menu (106) and by operation of the asthma condition assessment module (105) saved in the system (1) associated with the account (63) of the first user (33)(patient (33A). As to embodiments of the peak flow meter (52) or spirometer (51) which cannot be communicatively paired with the computing device (8A) the first user (33)(patient (33A) by user command (43) can enter the peak flow metric (116) into the peak flow field (119) and by operation of the asthma condition assessment module (105) saved in the asthma assessment device memory element (120) communicatively coupled to the asthma assessment device processor (121) and transmitted to the system (1) and associated with the account of the first user (63). While the asthma condition assessment menu (106) shown in the illustrative example of FIGS. 9 and 10 only shows a peak flow field (110) for entry of a peak flow metric (116) obtained by the first user's (33) performance of the peak flow test (111); this is not intended to preclude embodiments of the asthma condition assessment module (105) which functions to receive indications of asthma condition (48) based on other lung function tests (50), as above described, and functions to depict asthma condition metric fields (122) corresponding to the additional lung function tests (50) which can be filled with asthma condition metrics (47) generated by the corresponding asthma condition assessment devices (46) whether automatically by communicative pairing of the computing device (8) or manually by user command (43).

### Asthma Condition Tracking Module.

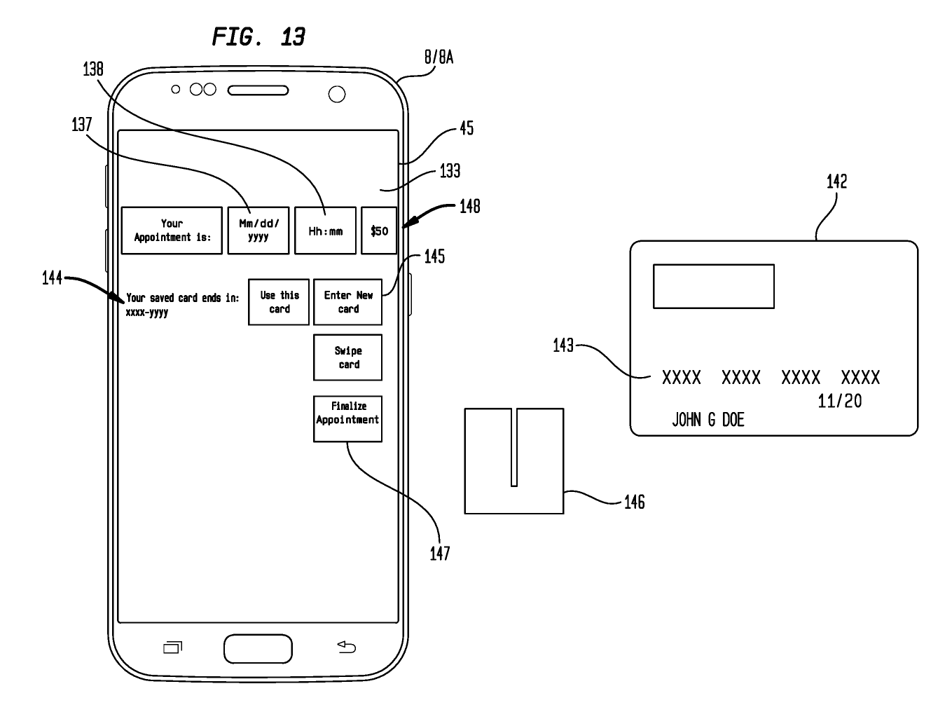

Now referring primarily to FIGS. 2 and 10 through 11, embodiments of the program (28) can, but need not necessarily, include an asthma condition tracking module (123) executable to compare indications of an asthma condition (48) of a first user (8A) based on one or more of: asthma condition metrics (47) or asthma condition assessment questionnaire score (108), stored in the system (1) over a duration of time. As one illustrative example, the asthma condition tracking module (123) can compare the asthma condition metric (47), such as the peak flow metric (116) corresponding to the peak flow (118) measured by a flow meter (52) or spirometer (51)(or other asthma assessment device (46)) at the current time, to all the prior asthma condition metrics (47), such as peak flow metrics (116) prior saved in association with the first user's (8) account (63) in the system (1). Now referring primarily to FIG. 10, as to particular embodiments the asthma condition tracking module (123) can be further executable to depict an asthma condition tracking menu (127) which can graphically depict asthma condition metrics (47) or asthma condition assessment questionnaire scores (108)(or both as shown in FIG. 10) over a duration of time. As to particular embodiments the asthma condition tracking module can function to provide a graph which illustrates change in asthma condition metrics (47) or asthma condition assessment questionnaire scores (108) over time (for example: peak flow (118) over a duration of time) in the form of a asthma condition graph (128) such as a bar graph, line graph, pie chart, or the like (shown in the example of FIG. 13 as two discrete plots of the asthma condition metrics (47) or asthma condition assessment questionnaire scores (108) over a twelve month period of time.

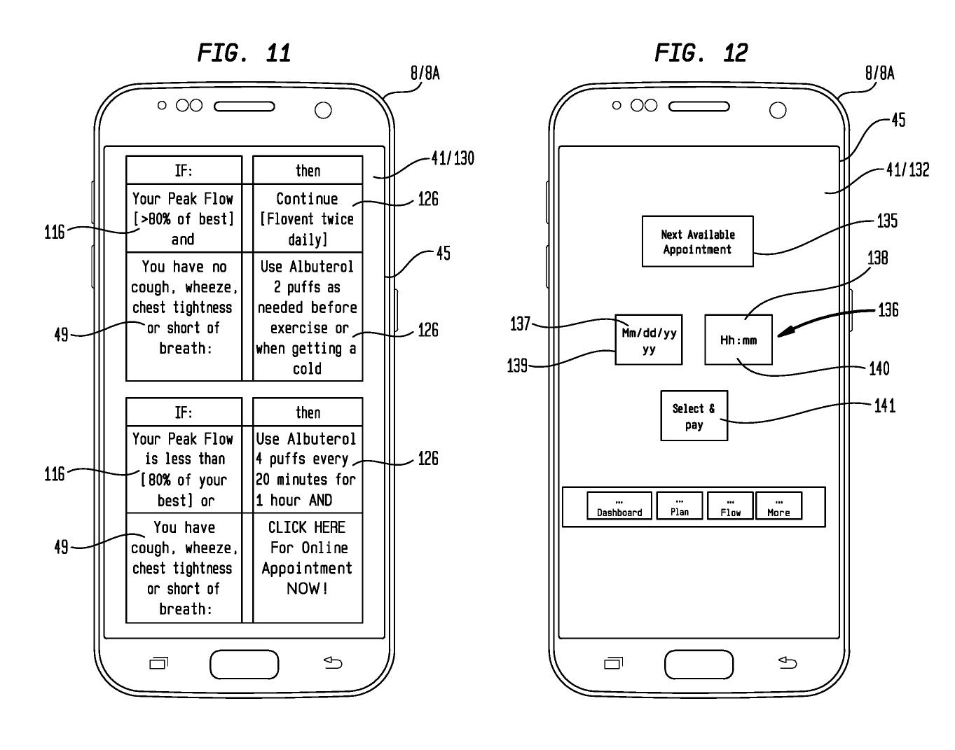

Now referring primarily to FIG. 11, based on the comparison performed by the asthma condition tracking module (123), as above described, the asthma condition tracking module (123) can be further executed to identify and depict on the display surface (45) of the first computing device (8A) one or more prescriptions (126) of a plurality of prescriptions (124) stored in a prescriptions database (125)(as shown the example of FIG. 2). As an illustrative example, if a peak flow metric (116) corresponding to the peak flow (118) measured by a flow meter (52) or spirometer (51) at the current time is greater than 80% of the best peak flow metric (116) of all the peak flow metrics (116) prior saved in association with the first user's (8) account (63) in the system (1), then the asthma condition tracking module (123) can identify and depict on the display surface (45) of the first user's device (8A) one or more prescriptions (126) that match the result of greater than 80% of the best peak flow metric (116). Similarly, if the peak flow metric (116) corresponding to the peak flow (118) measured by a flow meter (52) or spirometer (51) at the current time is less than 80% of the best peak flow metric (116) of all the peak flow metrics (116) prior saved in association with first user's account (63) in the system (1), then the asthma condition tracking module (123) can identify one or more prescriptions (126) that match the result of less than 80% of the best peak flow metric (116).

Again, referring primarily to FIG. 11, the asthma condition tracking module (123) can further function to identify one or more prescriptions (126) of the plurality of prescriptions (124) in the prescription database (125) in view of a current asthma condition assessment questionnaire score (108), whether further in view of or independent of the above described comparison of asthma condition metrics (47) of a first user (33)(patient (33A)) to the prior asthma condition metrics (47) stored in the system (1). As an illustrative example, if a current asthma condition assessment questionnaire score (108) (whether an ACT score or an ACQ score) provides current indications of an asthma condition (49) which do not include cough, wheeze, chest tightness or shortness of breath, then the asthma condition tracking module (123) can identify one or more prescriptions (126) from a plurality of prescriptions (124) in the prescriptions database (125) matched to the current asthma condition assessment questionnaire score (108). Similarly, if the current asthma condition assessment questionnaire score (108) provides current indications of an asthma condition (49) which includes cough, wheeze, chest tightness or shortness of breath, then the asthma condition tracking module (123) can identify one or more prescriptions (126) from the plurality of prescriptions (124) in the prescriptions database (125) matched to that current indications of an asthma condition (49).

### The Prescription Module

Now referring primarily to FIGS. 2 and 11, embodiments of the program (28) can, but need not necessarily, include a prescription module (129) which functions to depict prescription menu (130) on the display surface (45) of the first computing device (8A) which depicts the one or more prescriptions (126) identified by the asthma condition tracking module (123) based on comparison of asthma condition metrics (116) over a duration of time. As an illustrative example, the prescription module (129) can function to depict in the prescription menu (130) for a current peak flow metric (116) greater than 80% of the best prior peak flow metric (116) saved in association with the first user's account (63) in the system (1) can state that “Your peak flow is >80% of the best peak flow in your medical record, and depict a prescription (126) continue FLOVENT' twice daily.” Similarly, the asthma treatment prescription (126) depicted in the asthma prescription menu (130) for a peak flow less than 80% of the best peak flow metric (116) of all the peak flow metrics (116) prior saved in the first user account (63) in the system (1) can for example state “Use ALBUTEROL® 4 puffs every 20 minutes for 1 hour.”

Again, referring primarily to FIG. 11, the prescription module (129) can further function to depict the prescription menu (130) on the display surface (45) of the first computing device (8A) which depicts the one or more prescriptions (126) identified by the asthma condition tracking module (123) based on a current asthma condition assessment questionnaire score (108). The prescription module (129) can depict an asthma treatment prescription (126) based upon a current asthma condition assessment questionnaire score (108) which does not indicate any cough, wheeze, chest tightness or shortness of breath an asthma treatment prescription (126) stating “Use ALBUTEROL® 2 puffs as needed before exercise or when getting cold.” The asthma treatment prescription (126) depicted in the asthma condition tracking menu (130) based upon a current asthma condition assessment questionnaire score (108) which does indicate a cough, wheeze, chest tightness or shortness of breath, may not depict a prescription (126) but indicate “click here for on-line appointment NOW!”

### The Clinician Appointment and Payment Module.

Now referring primarily to FIGS. 2 and 12 through 13, embodiments of the program (1) can, but need not necessarily, further include a clinician appointment and payment module (131) executable to depict an appointment menu (132)(as shown in the example of FIG. 12) and a payment menu (1)(as shown in the example of FIG. 13) on the display surface (45) of the first computing device (8A). Now referring primarily to FIG. 2, the clinician appointment and payment module (131) receives indications of clinician appointments (134) and indications of payments for clinician services (134)(as shown in the example of FIG. 2). As shown in the illustrative example of FIG. 12, the clinician appointment and payment module (131) functions to depict an appointment icon (135)(shown as a “Schedule Appointment” button in the in the asthma condition assessment menu (132)) which by user command (43) can cause the clinician appointment and payment module (131) to identify the next available clinician appointment (136) and depict the appointment date and appointment time (137)(138) in respective appointment date and appointment time fields (139)(140)(as shown in the illustrative example of FIG. 12). As to particular embodiments, the clinician appointment and payment module (131) can further function to depict a next appointment icon (135)(show in the illustrative example of FIG. 12 as a “Next Available Appointment” button) in the appointment menu (132) which by serial user command (43) causes the clinician appointment and payment module (131) to correspondingly serially depict the appointment dates and appointments times (137)(138) in the respective appointment date and appointment time fields (139)(140). The first user (8A) can by user command (43) select and pay for the appointment date and appointment time (137)(138) with a second user (34)(clinician (34A). As shown by the illustrative example of FIG. 12, the first user (33)(patient 33A) interacts with an appointment selection and payment icon (141) which causes the clinician appointment and payment module (131) to further depict a payment menu (133) which allows a patient (33A) to pay for the clinician appointment (136) by credit or debit card (142). The a payment menu (133) allows by user command (43) entry of a credit or debit card number (143) in payment of the clinician appointment (136)(as shown in the example of FIG. 13, entry of the credit or debit card number (43) can be by selection of a card number from a list of card numbers (144) associated with the account (63) of the first user (8A) or by entry of the card number (143) into a card number field (145) or by card swipe or insertion into a credit card reader (146). As to particular embodiments the first user (33)(patient (33A)) can interact with an appointment confirmation icon (147) to cause the clinician appointment and payment module (131) to depict an appointment confirmation field (148) including the clinician appointment date and appointment time (137)(138).

### The Clinician Module.

Now referring primarily to FIGS. 2 and 14 through 15, embodiments of the program (28) can, but need not necessarily include, a clinician module (148) which can be downloaded from the server (2) to a second computing device (8B) used by a second user (34)(which can be a clinician (34A). The clinician module (148) can be executed to depict the sign up and log in menus (62)(65) (as shown in the example of FIG. 3) which by user command (43) allows the clinician (34A) to establish a clinician account (149) in the system (1), in similar manner to above described for the first user (33)(patient (33A)). The clinician module (148) can be further executed to depict a clinician menu (150) on the display surface (45) of the second computing device (8B). The clinician module (148) can be further executed to identify new patients (151) that have paid for clinician appointments (136) and pair the patient account (63) to a clinician account (149).

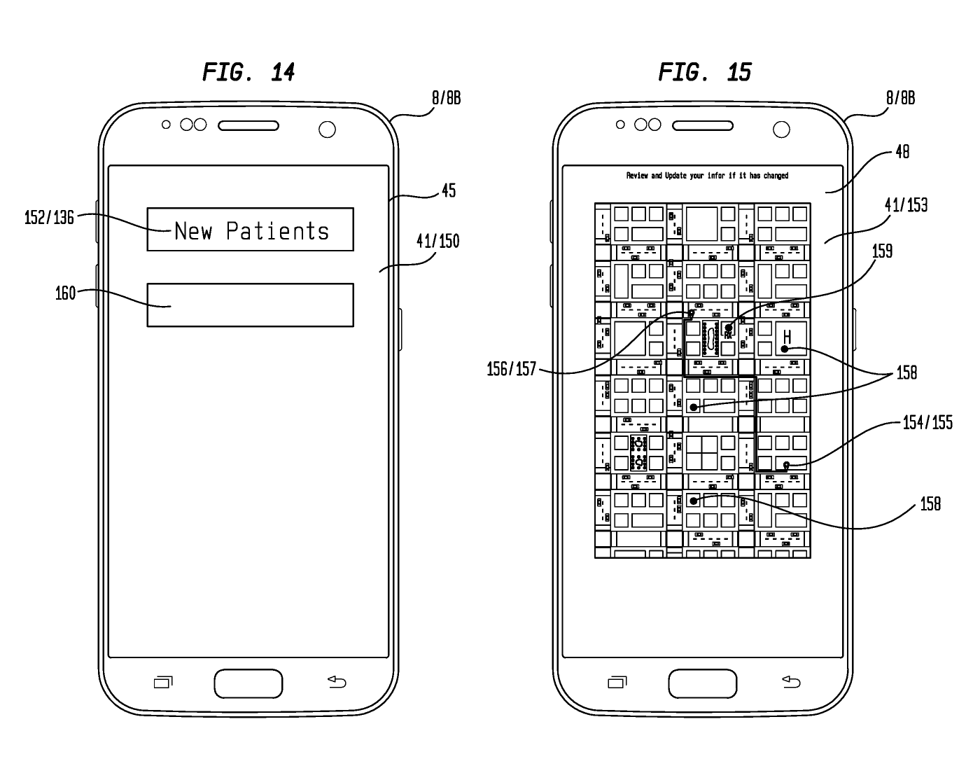

Now referring primarily to FIG. 14, the clinician module (148) can be further executed to depict the clinician menu (150) on the display surface (45) of the second computing device (8B). The clinician module (148) can be further executed to depict in the clinician menu (150) one or more appointment prompts (152) corresponding to clinician appointments (136) made in patient accounts (63) paired with the clinician account (149). The clinician (34A) by user command (43) can interact with one of the appoint prompts (152) to access the system (1) to obtain one or more of: the associated patient profile (95), released medical records (103) and indications of asthma condition (49) of the patient (33A), each as above described. As shown in the illustrative example of FIG. 14 the appointment prompt (152) can be via any one or more of the communication resources (16) supported by the network (9) such as text message or electronic mail.

Now referring to FIG. 15, as to particular embodiments, in which the GPS application (18) has been downloaded, the clinician module (150) can depict a geo-spatial location menu (153) in which the current location (154) at a current time (155) or an expected future location (156) at a future time (157) of a patient (33A) can be identified along with other geographic features, as illustrative examples: elevation, latitude, longitude; locations, as illustrative examples: hospitals, fire station, urgent care centers, drug stores; or conditions, as illustrative examples: temperature, humidity, pollen count, allergens, relevant to treatment of indications of an asthma condition (48).

The clinician module (148) further functions to automatically contact the patient (33A) prior to the appointment date and time (137)(138) or depicts in the clinician menu (150) patient contact information (160) of the patient (33A). The clinician can contact the patient (33A) and advises the patient (33A) on the current indications of asthma condition (48), which can include as illustrative examples one or more of: assessing the status of asthma disease management; providing recommendations for patient self-management; revising the asthma condition prescription (126) which can be depicted in the asthma condition prescription menu (130) of the first computing device (8A) used by the patient (33A); scheduling a follow-up appointment in approximately 3 months which can be depicted in the an appointment and payment menu (132) of the first computing device (8A) used by the patient (33A).

As to particular embodiments, the clinician module (148) can further function to depict a prescription ordering menu (161) which allows the clinician (34A) by user command (43) to enter prescriptions (126) to treat the indications of asthma condition (48) and which can be depicted in the asthma condition prescription menu (130) of the first computing device (8A). As to particular embodiments, the prescription (126) can be automatically transmitted to a pharmacy location (159)(as shown in the example of FIG. 15) and as to particular embodiments transmitted to a pharmacy (160) based on the current location (154) or the expected future location (156) of the patient (33A).

As can be easily understood from the foregoing, the basic concepts of the present invention may be embodied in a variety of ways. The invention involves numerous and varied embodiments of distant asthma assessment and prescription system (1) and methods for making and using such distant asthma assessment and prescription system (1) including the best mode.

As such, the particular embodiments or elements of the invention disclosed by the description or shown in the figures or tables accompanying this application are not intended to be limiting, but rather exemplary of the numerous and varied embodiments generically encompassed by the invention or equivalents encompassed with respect to any particular element thereof. In addition, the specific description of a single embodiment or element of the invention may not explicitly describe all embodiments or elements possible; many alternatives are implicitly disclosed by the description and figures.

It should be understood that each element of an apparatus or each step of a method may be described by an apparatus term or method term. Such terms can be substituted where desired to make explicit the implicitly broad coverage to which this invention is entitled. As but one example, it should be understood that all steps of a method may be disclosed as an action, a means for taking that action, or as an element which causes that action. Similarly, each element of an apparatus may be disclosed as the physical element or the action which that physical element facilitates. As but one example, the disclosure of a “indications of an asthma condition” should be understood to encompass disclosure of the act of “indicating an asthma condition”—whether explicitly discussed or not—and, conversely, were there effectively disclosure of the act of “indicating an asthma condition”, such a disclosure should be understood to encompass disclosure of a “indications of an asthma condition” and even a “means for indicating an asthma condition.” Such alternative terms for each element or step are to be understood to be explicitly included in the description.

In addition, as to each term used it should be understood that unless its utilization in this application is inconsistent with such interpretation, common dictionary definitions should be understood to be included in the description for each term as contained in the Random House Webster's Unabridged Dictionary, second edition, each definition hereby incorporated by reference.

All numeric values herein are assumed to be modified by the term “about”, whether or not explicitly indicated. For the purposes of the present invention, ranges may be expressed as from “about” one particular value to “about” another particular value. When such a range is expressed, another embodiment includes from the one particular value to the other particular value. The recitation of numerical ranges by endpoints includes all the numeric values subsumed within that range. A numerical range of one to five includes for example the numeric values 1, 1.5, 2, 2.75, 3, 3.80, 4, 5, and so forth. It will be further understood that the endpoints of each of the ranges are significant both in relation to the other endpoint, and independently of the other endpoint. When a value is expressed as an approximation by use of the antecedent “about,” it will be understood that the particular value forms another embodiment. The term “about” generally refers to a range of numeric values that one of skill in the art would consider equivalent to the recited numeric value or having the same function or result. Similarly, the antecedent “substantially” means largely, but not wholly, the same form, manner or degree and the particular element will have a range of configurations as a person of ordinary skill in the art would consider as having the same function or result. When a particular element is expressed as an approximation by use of the antecedent “substantially,” it will be understood that the particular element forms another embodiment.

Moreover, for the purposes of the present invention, the term “a” or “an” entity refers to one or more of that entity unless otherwise limited. As such, the terms “a” or “an”, “one or more” and “at least one” can be used interchangeably herein.

Thus, the applicant(s) should be understood to claim at least: i) each embodiment of the treatment control system herein disclosed and described, ii) the related methods disclosed and described, iii) similar, equivalent, and even implicit variations of each of these devices and methods, iv) those alternative embodiments which accomplish each of the functions shown, disclosed, or described, v) those alternative designs and methods which accomplish each of the functions shown as are implicit to accomplish that which is disclosed and described, vi) each feature, component, and step shown as separate and independent inventions, vii) the applications enhanced by the various systems or components disclosed, viii) the resulting products produced by such systems or components, ix) methods and apparatuses substantially as described hereinbefore and with reference to any of the accompanying examples, x) the various combinations and permutations of each of the previous elements disclosed.

The background section of this patent application provides a statement of the field of endeavor to which the invention pertains. This section may also incorporate or contain paraphrasing of certain United States patents, patent applications, publications, or subject matter of the claimed invention useful in relating information, problems, or concerns about the state of technology to which the invention is drawn toward. It is not intended that any United States patent, patent application, publication, statement or other information cited or incorporated herein be interpreted, construed or deemed to be admitted as prior art with respect to the invention.

The claims set forth in this specification, if any, are hereby incorporated by reference as part of this description of the invention, and the applicant expressly reserves the right to use all of or a portion of such incorporated content of such claims as additional description to support any of or all of the claims or any element or component thereof, and the applicant further expressly reserves the right to move any portion of or all of the incorporated content of such claims or any element or component thereof from the description into the claims or vice-versa as necessary to define the matter for which protection is sought by this application or by any subsequent application or continuation, division, or continuation-in-part application thereof, or to obtain any benefit of, reduction in fees pursuant to, or to comply with the patent laws, rules, or regulations of any country or treaty, and such content incorporated by reference shall survive during the entire pendency of this application including any subsequent continuation, division, or continuation-in-part application thereof or any reissue or extension thereon.

Additionally, the claims set forth in this specification, if any, are further intended to describe the metes and bounds of a limited number of the preferred embodiments of the invention and are not to be construed as the broadest embodiment of the invention or a complete listing of embodiments of the invention that may be claimed. The applicant does not waive any right to develop further claims based upon the description set forth above as a part of any continuation, division, or continuation-in-part, or similar application.

1-15. (canceled)

16. A computer implemented system, comprising:
an asthma condition assessment device which generates at least one air capacity measurement of lungs;
a client computing device including a processor communicatively coupled to a memory element, said memory element containing a computer readable program code executable by said processor to:
depict an asthma condition assessment menu on the display surface of said client computing device, said asthma condition assessment menu adapted to receive asthma condition indications correlatable with an asthma condition;
receive asthma condition indications in said asthma condition assessment menu;
transmit asthma condition indications for correlation of said asthma condition indications with said asthma condition; and
receive a prescription to treat said asthma condition based on correlation of said asthma condition indications to said asthma condition; and
depict said prescription on the display surface of said client computing device.

17. The system of claim 16, further comprising generate asthma condition metrics based upon said at least one air capacity measurement of lungs.

18. The system of claim 17, wherein said at least one air capacity measurement of lungs comprises at least one air flow measurement generated by said asthma condition assessment device subsequent to performance of a lung function test.

19. The system of claim 18, wherein said air flow measurement is selected from the group consisting of: a forced vital capacity, forced expiratory volume and maximal voluntary ventilation, or combinations thereof.

20. The system of claim 17, further comprising depict an asthma condition assessment questionnaire adapted to receive asthma condition indications entered by a client.

21. The system of claim 20, wherein said asthma condition metrics comprise an asthma condition assessment questionnaire score based on said indications of asthma condition entered into said asthma condition assessment questionnaire.

22. The system of claim 16, wherein said asthma condition assessment device further comprises a transmitter adapted to transmit at least one air capacity measurement and wherein said client computing device further comprises a receiver adapted to receive said at least one air capacity measurement from said asthma condition assessment device, said computer readable program further executable to receive said at least one air capacity measurement as said asthma condition indications correlatable with said asthma condition.

23. The system of claim 22, further comprising communicatively pair said asthma condition assessment device to said client computing device.

24. The system of claim 23, wherein said transmitter comprises one or more of a BLUETOOTH® or WI-FI® transmitter and said receiver comprises one or more of a BLUETOOTH® or WI-FI® receiver.

25. The system of claim 16, wherein said prescription received by said client computing device to treat said asthma condition is based on a calculated difference between current asthma condition metrics transmitted by said client computing device and prior asthma condition metrics transmitted by said client device.

26. The system of claim 25, wherein said asthma condition metrics comprise air flow measurements transmitted by said client computing device, and wherein a current air flow measurement transmitted by said client computer and prior air flow measurements transmitted by said client computer within a period of time allow calculation of a percentage based on the quotient of said current air flow measurement over the greatest said prior air flow measurement transmitted in said period of time, said prescription received by said client computer to treat said asthma condition matched to said percentage.

27. The system of claim 16, wherein said client computing device comprises a mobile computing device, and further comprising:
transmit a current location of said mobile computing device at a current time;
receive said prescription to treat said asthma condition based on correlation of said one or more environmental conditions associated with said current location of said mobile device at said current time and said asthma condition indications transmitted from said mobile computing device at said current location at said current time.

28. The system of claim 16, further comprising:
transmit an expected future location of said mobile computing device at a future time;
receive said prescription to treat said asthma condition based on correlation of said environmental conditions associated with said expected future location of said mobile device at said future time and said asthma condition indications obtained from said mobile computing device at said current location at said current time.

29. The system of any one of claim 27 or 28, wherein said environmental conditions associated with said location of said mobile device is selected from the group consisting of: an elevation, a latitude, a longitude, a temperature, a humidity, a pollen count, an allergen, or a combination thereof.

30. The system of any one of claim 27 or 28, further comprising receive identified prescription fulfilment locations based on said prescription and said current location of said mobile computing device at said current time or based on said prescription and said expected future location of said mobile computing device at said future time.

31. The system of any one of claim 27 or 28, further comprising receive identified asthma condition treatment locations based on said asthma condition indications received from a client computing device and said current location of said mobile computing device at said current time or based said expected future location of said mobile computing device at said future time.

32. The system of claim 16, further comprising:
receive indications of available clinician appointments;
transmit selection of one of said available clinician appointments;
receive indications confirming a clinician appointment; and
transmit payment for said clinician appointment.

33-58. (canceled)
